(19) United States
(12) Patent Application Publication
(10) Pub. No.: US 2011/0223193 A1
(43) Pub. Date: Sep. 15, 2011

(54) METHOD FOR THE PREPARATION OF MEDICAL DEVICES AND DEVICES OBTAINED THEREFROM

(75) Inventor: Valentino Mercati, Sansepolcro (IT)

(73) Assignee: ABOCA S.P.A. SOCIETA ARGRICOLA, Sansepolcro (Arezzo) (IT)

(21) Appl. No.: 13/128,322

(22) PCT Filed: Nov. 10, 2009
(86) PCT No.: PCT/IB09/07403

§ 371 Date: Jun. 2, 2011

(30) Foreign Application Priority Data
Nov. 10, 2008 (IT) MI2008A1974

(51) Int. Cl. A61K 36/00; A61K 8/97; A61K 36/73; A61K 36/185; A61K 36/45; A61K 36/49; A61K 36/328; A61K 36/38; A61K 36/53; A61K 36/734; A61K 36/738; A61K 36/14; A61K 36/28; A61K 36/288; A61K 31/702; A61K 31/715; A61K 36/68; A61K 36/55; A61K 36/8998; A61K 36/54; A61P 1/00; A61P 31/00; A61P 33/00; A61P 17/00; A61Q 19/00; A61P 29/00; A61P 17/02; A61P 39/00

(52) U.S. Cl. 424/195.18; 424/725; 424/765; 424/774; 424/775; 424/771; 424/748; 424/730; 424/732; 424/745; 424/777; 424/769; 424/776; 424/764; 514/23; 514/54; 424/738; 424/773; 424/768; 424/739

(57) ABSTRACT

Method of selecting and preparing medical devices comprising extracts of plants and natural substances devoid of pharmacologically, immunologically or metabolically active fractions and characterised by a content of substances able to exert beneficial effects on the body, mediated by mechanical or physical effects, which said process comprises:
a) removal by conventional processes of any pharmacologically, immunologically or metabolically active fractions from extracts of plants or natural substances;
b) biological testing to establish that the extract or substance obtained in a) is devoid of pharmacological, metabolic and immunological effects;
c) formulation of the extracts or substances that pass the test conducted at stage b) into suitable medical devices.

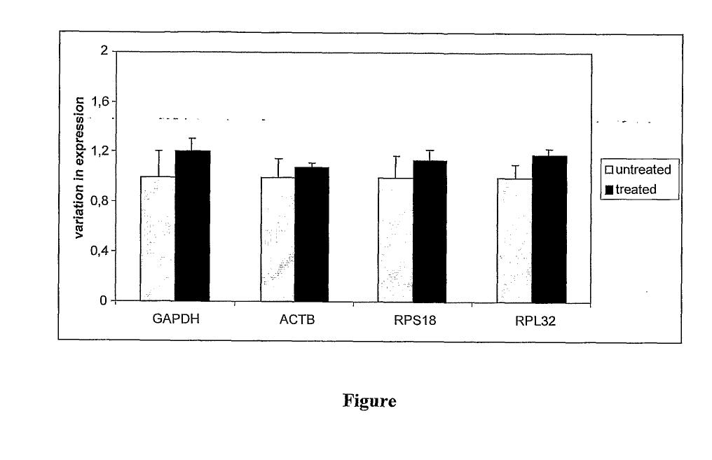

This invention relates to a method for the selection and preparation of medical devices comprising extracts of plants or natural substances containing no pharmacologically, immunologically or metabolically active fractions and characterised by a content of substances able to exercise effects on the body that are mediated by chemico-physical, mechanical or physical effects, which said process comprises:

a) removal by conventional processes of any pharmacologically, immunologically or metabolically active fractions from extracts of plants or natural substances;

b) biological testing to establish that the extract or substance obtained in a) is devoid of pharmacological, metabolic or immunological effects;

c) formulation of the extracts or substances that pass the test conducted at stage b) into suitable medical devices.

The medical devices thus obtained can have a moisturising, soothing, adsorbent, lubricating, occlusive/barrier effect, and/or neutralising, abrasive, surfactant or antimicrobial activity. The compositions according to the invention alleviate or prevent irritant symptoms at regional level in various parts of the body. The beneficial effect of said compositions on the body is not due to a pharmacological, metabolic or immunological action mechanism.

### PRIOR ART

Historically, humans have always sought to obtain health benefits from matrices belonging to the plant, animal or mineral kingdom. These matrices were employed to make preparations used as medicinal products, and it is only as a result of the studies conducted in the last century that researchers have in many cases come to understand the chemical structures of the substances responsible for their therapeutic activity.

In particular, compounds present in matrices which interact with the physiological and biological processes of the body, mainly by means of interactions with macromolecular components such as receptors, enzymes or other substances present in the organs and tissues, were classified as active according to the principles of conventional pharmacology.

From the quantitative standpoint, said active compounds are always contained in the biological matrices in minimal quantities, the remainder being considered substantially useless, or at most an adjuvant to the pharmacological action attributed to the active fraction. Significantly, current research in the field of phytotherapy tends to concentrate and/or isolate the active substances or fractions in accordance with the mechanisms of conventional pharmacology, and minimise the presence of other components which are considered “inert” because they do not activate receptor or immune pathways or metabolic processes.

Technologies involving extraction and fractionation of the single molecules and/or fractions of the natural complex are increasingly evolving for this purpose: apart from the usual column fractionation, researchers have recently described membrane filtration techniques such as microfiltration and ultrafiltration, molecular distillation, ultrasound extraction, extraction with supercritical CO₂, and fractionation with special solvents and/or reagents, using differential temperatures (e.g. fractionated distillation), with separation of lipophilic and hydrophilic substances and selection based on pH.

Said techniques are designed to isolate from the natural complexes the pharmacologically active fractions and/or molecules, ie. those able to modify the metabolic, physiological and biological processes of the living body by direct molecular interaction.

The most common active substances belong to different classes, such as alkaloids, flavonoids, glucosides, terpenes, carotenoids, vitamins, etc.

The therapeutic potential of plant matrices on the body performed by means of a mechanical action, ie. an action mainly due to the part which has so far been considered inactive, has never been exploited.

Said “mechanical” action could be advantageously used to prepare medical devices, defined in Directive EEC 93/42 as “any instrument, apparatus, appliance, material or other article, whether used alone or in combination . . . intended . . . to be used for human beings for the purpose of diagnosis, prevention, monitoring, treatment or alleviation of disease; diagnosis, monitoring, treatment, alleviation of or compensation for an injury or handicap; investigation, replacement or modification of the anatomy or of a physiological process; control of conception; and which does not achieve its principal intended action in or on the human body by pharmacological, immunological or metabolic means, but which may be assisted in its function by such means”.

The importance that medical devices can have in the healthcare field is therefore obvious. A method of preparing said devices which allows a distinction to be made between (i) the specifically pharmacological, immunological and metabolic effects characteristic of drugs, and (ii) other types of effect on the body which may fall within the definition of said EEC Directive, is therefore desirable.

### DESCRIPTION OF THE INVENTION

It has now been found, and this finding constitutes the subject of the invention, that the substances in said conventional phytotherapeutic matrices which have been considered inactive to date (and fit only to be discarded, or at most used as a vehicle for the active substances) actually have an interesting and advantageous therapeutic potential due to non-pharmacological interactions with the tissues and organs of animal bodies, in particular those of the human body, said interactions being due to chemical/physical or mechanical action mechanisms and/or regulation/repair of altered physiological conditions at regional and local level, without any involvement by strictly metabolic, pharmacological or immunological bodily processes.

In particular, it has been found that plant matrices or extracts from which the pharmacologically active fractions or substances have been removed or neutralised present a content (often preponderant) of substances which still perform a therapeutic/health function due to a prevalent “physical or mechanical action” at local level.

The first aspect of this invention therefore relates to a method of selecting and preparing medical devices comprising extracts of plants and natural substances devoid of pharmacologically, immunologically or metabolically active fractions and characterised by a content of substances able to exert beneficial effects on the body, mediated by mechanical or physical effects, which said process comprises:

a) removal by conventional processes of any pharmacologically, immunologically or metabolically active fractions from extracts of plants or natural substances;

b) biological testing to establish that the extracts or substances obtained in a) are devoid of pharmacological, metabolic or immunological effects;

c) formulation of the extracts or substances that pass the test conducted at stage b) into suitable medical devices.

Said non-pharmacological interactions, as already stated, are due to a substantially physical or mechanical mechanism and/or regulation/repair at topical level of physiological conditions such as the pH of tissues and mucous membranes.

Examples of said non-pharmacological interactions include:
Impermeabilisation and/or protection of mucous membranes with gastroprotective, antiparasitic and/or anti-infectious effects (e.g. protection against lice, herpes, etc.);
Inhibition of the growth and spread of micro-organisms;
Surfactant action;
Adsorption or chelation of exogenous or endogenous irritant or pathogenic substances with an anti-gas, soothing and/or protective effect;
Adsorption or chelation of water or nutrients with an effect on peristalsis and weight;
pH-regulating action;
Water supply to the skin, organs (e.g. the intestine) or mucous membranes, with a moisturising, softening/emulsifying and lubricant effect;
Reduction of friction between mucous membranes or between external agents and the mucous membranes or skin, with a lubricant and soothing effect;
Action on temperature with a refreshing/analgesic or massage effect;
Filling action (constitution of mechanical substrates with natural polymers such as resins and polysaccharides) with a wound-healing and anti-wrinkle effect and a support effect for nails and hair;
Interaction with solar radiation with a protective and soothing effect;
Irritant action with a rubefacient, cathartic effect, and a “scrub” effect (with removal of incrustations and debris from tissues in the gastroenteric, ENT and dermatological spheres);
A direct and/or indirect effect on the viscoelastic characteristics of the mucus with a mucolytic/fluidifying effect at gastroenteric, ENT and dermatological level.

The substances able to exercise effects on the body mediated by mechanical or physical effects include tannins, soluble or insoluble fibres, polysaccharides, mucilages, resins, lipids, waxes, triglycerides and fatty acids, suitably processed.

Tannins can be extracted from the following plants (the preferred parts of the plant are shown in brackets): potentilla (rhizome), witch-hazel (leaves and bark), bearberry (leaves), oak (leaves, bark and oak gall), Gallic rose (flowers), lady's mantle (leaves and flowers), agrimony (herb), myrrh, St. John's wort (leaves and flowers), bilberry (leaves and berries), vine (leaves and seeds), thyme (herb), blackcurrant (leaves and berries), hawthorn (leaves, flowers and berries) and cypress (fruit).

Soluble or insoluble fibres can be extracted from the following plants (the preferred parts of the plant are shown in brackets): opunzia (cladophylls), chicory (root), dandelion (root) and burdock (root).

Polysaccharides/oligosaccharides can be extracted from the following plants (the preferred parts of the plant are shown in brackets): aloe (leaf gel, leaves), sage (leaves), camomile (flowers), plantain (leaves, herb), calendula (flowers), fenugreek (seed), thyme (herb), gentian (root), liquorice (root), lichen (thallus), devil's claw (root), turmeric (root), Echinacea (root), Ginkgo biloba (leaves), Fucus (thallus), ginseng (root), Eleutherococcus (root), caraway (seed), goldenrod (herb), everlasting flower (flower), blackcurrant (leaves and berries), glucomannan (tuber) and opunzia (cladophylls). Gums and mucilages can be extracted from the following plants (the preferred parts of the plant are shown in brackets): French/blond psyllium (Plantago arenaria/Plantago ovata, seed), ribwort plantain (Plantago lanceolata), mallow (leaves, flowers), marshmallow (root), linden (flowers and leaves), flax (seed), barley (seed), senna (leaves and fruit), borage (leaves), cinnamon (bark), elder (flowers), tragacanth (gum), Sterculia (gum), acacia (gum), guar (gum) and xanthan (gum).

Terpene and phenol resins, oleoresins and gum resins can be extracted from the following plants or materials of plant origin, possibly processed by animals (the preferred parts of the plant are shown in brackets): propolis, benzoin, turpentine, balsam of Peru, balsam of Tolu, paprika, lentisk or mastic tree, myrrh, incense, ginger (root), caraway (seeds), Grindelia (flower head), asafoetida and lignum vitae.

Lipids, triglycerides or waxes can be extracted from the following plants (the preferred parts of the plant are shown in brackets): cocoa (seed), flax (seed), borage (seed), evening primrose (seed), argan (berries), almond (seed), wheat (seed), hemp (seed) and blackcurrant (seed).

Fatty acids are extracted from the following plants (the preferred parts of the plant are shown in brackets): anise (seed), caraway (fruit), cardamom (seed), coriander (seed), fennel (seed), blessed milk thistle (seed), everlasting flower (flower head) and arnica (flower).

The techniques used to remove the pharmacologically active compounds or fractions from said plant sources are well-known, and in any event accessible by one skilled in the art.

The biological tests used at stage b) are also known, such as the housekeeping gene expression test or the test for inhibition of release of inflammation mediators such as interleukin 6 and interleukin-8 from cultured cells such as fibroblasts.

The benefits of a mechanical action without pharmacological interference by other substances can be very important to prevent systemic interactions with pharmacologically active substances. The devices according to the invention also meet the requirements of European Directive 93/42 EEC, transposed by each member state according to its national legislation.

The invention will be now be described in greater detail by means of the following Examples.

### Example 1

### Sage

The main constituents of Salvia officinalis leaves are essential oil, tannins, flavonoids and polysaccharides.

Extracts mainly containing polysaccharides or fractions of sage extracts enriched (ie. concentrated to the exclusion of other substances) with polysaccharides perform a therapeutic/health function due to the prevalent “mechanical action” with a barrier effect and/or occlusive effect.

### Example 2

### Senna

The main constituents of senna leaves are anthraquinone glycosides (sennosides), flavonoids, mucilages and essential oil.

At present, the metabolic activities of senna are mainly attributed to anthraquinone glycosides. Extracts mainly containing mucilage or fractions of senna extracts enriched with mucilage perform a therapeutic/health function due to a “mechanical action” with an adsorbent and/or barrier effect and/or lubricant effect.

### Example 3

### Propolis

The main constituents of propolis are resin, wax, volatile substances, aromatic acids, flavonoids and minerals.

At present, the metabolic activities of propolis are mainly attributed to aromatic acids and flavonoids. Extracts mainly containing resins or fractions of propolis extracts enriched with resins perform a therapeutic/health function due to a “mechanical action” with a barrier effect and/or occlusive effect.

### Example 4

### Camomile

The main constituents of Matricaria chamomilla flowers are essential oil, sesquiterpene lactones, flavonoids and mucilages.

Extracts mainly containing mucilages or fractions of camomile extracts enriched with mucilages perform a therapeutic/health function due to a “mechanical action” with a barrier effect and/or occlusive effect.

### Example 5

### Equisetum

The main constituents of equisetum herb are inorganic salts, flavonoids, dicarboxylic acids and mucilages.

Extracts mainly containing inorganic salts or fractions of equisetum extracts enriched with inorganic salts perform a therapeutic/health function due to a “mechanical action” with a barrier effect and/or occlusive effect and/or neutralising effect.

### Example 6

### Medical Device Designed to Protect the Gastric Mucosa

A study was conducted on the action of a plant preparation based on natural carbonates and polysaccharides designed to protect the gastric mucosa by means of a local buffer action mechanism which has a first direct, mechanical action, constituting a mechanical barrier and a chemical barrier against acid attack.

The study was based on gene expression by a group of four “housekeeping” genes, ie. genes expressed in all human tissues and cell types. The four genes encode four proteins constitutionally present in all body cells. The expression of said genes in a cell line of gastric origin was evaluated in particular, before and after treatment with the product. For each of the four genes, the expression values of the treated cells were compared with the expression value of the untreated cells.

Analysis of the results shown in the Figure demonstrates that treatment with the product does not significantly change the expression of the housekeeping genes tested, and that its action can therefore be considered purely mechanical. The protective activity of the product on the gastric mucosa was also confirmed on experimental animal models of lesions of the gastric mucosa. The experimental method rules out an action due to pharmacological or metabolic effects, and confirms that the action observed meets the definitions of medical devices set out in Directive EEC 93/42.

Example of Composition

| constituent | g/kg | |
|---|---|---|
| CALCIUM CARBONATE | 15-35 | |
| SODIUM BICARBONATE | 1-10 | |
| MARSHMALLOW ROOT MUCILAGES | 0.5-10 | |
| BARLEY MUCILAGES | 0.5-10 | |
| ALOE VERA dried extract 200:1 | 0.5-10 | |
| CAMOMILE LIGULE freeze-dried extract | 0.5-10 | |
| SPEARMINT FLAVOURING or other natural | | |
| flavourings | | |
| LIQUORICE freeze-dried extract | 0.5-10 | |
| SUGAR CANE | q.s. for 1000 | g |

### Example 7

### Preparation of a Medical Device Based on Honey, Resins and Polysaccharides as Adjuvant in Cough Treatment

The product creates a protective film with a “barrier effect” which calms the cough, protecting the pharynx and oesophagus against post-nasal drip or other irritant situations conducive to coughing.

Honey is used for its mechanical and mucoadhesive action which produces the barrier effect characteristic of the product.

The nutritional metabolic action of honey has no function or involvement in calming the cough.

A mechanical barrier effect test was conducted to establish the ability of the product to limit contact between the mucosa and external irritants. A portion of lipopolysaccharide from Escherichia coli cell membrane was used as irritant agent. The production of IL 6 and IL 8 as a result of contact between a layer of cells (fibroblasts) and the LPS was measured. IL-6 is more specific for this type of attack, so inhibition of IL-6 is the direct measurement of the barrier effect.

The results, conducted against a negative control, demonstrate that the presence of the product on a membrane placed above the cells reduces the production of IL 6 and IL 8 to a statistically significant extent.

This confirms the barrier effect of the product. The fact that such a reduction in cytokine production is due to a mechanical barrier effect and not to the anti-inflammatory action of the sample is demonstrated by direct comparison with the various positive controls, wherein the experimental membranes placed above the cells (and not in contact with them) have no coating of the product, and the allergen passing through the membrane without obstacles arrives at the culture medium and reaches concentrations able to sensitise the cells.

| constituent | g/1000 ml |
|---|---|
| Plantain leaves, freeze-dried extract | 3-12 |
| Grindelia tips, freeze-dried extract | 3-12 |
| Honey | 200-1200 |
| Everlasting flower tips, freeze-dried | 1-4 |
| extract | |
| Flavourings or essential oils | |

### LITERATURE

Trease and Evans Pharmacognosy, William Charles Evans (ed.), 15th Edition, W.B. Saunders, Edinburgh, UK, 2002.
Lezioni di Farmacognosia. Droghe vegetali, Fassina Giuliana-CEDAM-Padova, 1974.
Medicinal spices; a handbook of culinary herbs, spices, spice mixtures and their essential oils. Teuscher, Eberhard. Trans. by Josef A. Brinckmann & Michael P. Lindermaier. CRC Press, 2006.
Herbal Drugs and Phytopharmaceuticals, Third Edition Max Wichtl ed., CRC Press, 2004.
ESCOP 2003: ESCOP Monographs: The Scientific Foundation for Herbal Medicinal Products, 2nd edition. Exeter (UK): European Scientific Cooperative on Phytotherapy and Thieme; 2003.

1. Method of selecting and preparing medical devices comprising extracts of plants and natural substances devoid of pharmacologically, immunologically or metabolically active fractions and characterised by a content of substances able to exert a beneficial effect on the body mediated by mechanical or physical effects, said process comprising:
a) removing any pharmacologically, immunologically or metabolically active fractions from extracts of plants or natural substances;
b) biological testing to establish that the extract or substance obtained in a) is devoid of pharmacological, metabolic and immunological effects;
c) formulating the extracts or substances that pass the test conducted at stage b) into suitable medical devices.

2. Method as claimed in claim 1, for the preparation of medical devices having one of the following actions:
Impermeabilisation and/or protection of mucous membranes with gastroprotective, antiparasitic and/or anti-infectious effects;
Inhibition of the growth and spread of micro-organisms;
Surfactant action;
Adsorption or chelation of exogenous or endogenous irritant or pathogenic substances with an anti-gas, soothing and/or protective effect;
Adsorption or chelation of water or nutrients with an effect on peristalsis and weight;
pH-regulating action;
water supply to the skin, organs or mucous membranes with a moisturising, softening/emulsifying and lubricant effect;
Reduction of friction between mucous membranes or between external agents and the mucous membranes or skin, with a lubricant and soothing effect;
Action on temperature with a refreshing/analgesic or massage effect;
Filling action with a wound-healing and anti-wrinkle effect and a support effect for nails and hair;
Interaction with solar radiation with a protective and soothing effect;
Irritant action with a rubefacient, cathartic effect, and a “scrub” effect;
A direct and/or indirect effect on the viscoelastic characteristics of the mucus with a mucolytic/fluidifying effect at gastroenteric, ENT and dermatological level.

3. Method as claimed in claim 1, wherein the substances able to exercise effects on the body mediated by mechanical or physical effects include tannins, soluble or insoluble fibres, polysaccharides, mucilages, resins, lipids, waxes, triglycerides and fatty acids.

4. Method as claimed in claim 3, wherein the substances able to exercise effects on the body mediated by mechanical or physical effects are tannins.

5. Method as claimed in claim 4, wherein the tannins are extracted from plants chosen from potentilla (rhizome), witch-hazel (leaves and bark), bearberry (leaves), oak (leaves, bark and oak gall), Gallic rose (flowers), lady's mantle (leaves and flowers), agrimony (herb), myrrh, St. John's wort (leaves and flowers), bilberry (leaves and berries), vine (leaves and seeds), thyme (herb), blackcurrant (leaves and berries), hawthorn (leaves, flowers and berries), and cypress (fruit).

6. Method as claimed in claim 1, wherein the substances able to exercise effects on the body mediated by mechanical or physical effects are soluble or insoluble fibres.

7. Method as claimed in claim 6 wherein the fibres are extracted from plants chosen from opunzia (cladophylls), chicory (root), dandelion (root) and burdock (root).

8. Method as claimed in claim 1, wherein the substances able to exercise effects on the body mediated by mechanical or physical effects are polysaccharides/oligosaccharides.

9. Method as claimed in claim 8, wherein the polysaccharides/oligosaccharides are extracted from plants chosen from aloe (leaf gel, leaves), sage (leaves), camomile (flowers), plantain (leaves, herb), calendula (flowers), fenugreek (seed), thyme (herb), gentian (root), liquorice (root), lichen (thallus), devil's claw (root), turmeric (root), Echinacea (root), Ginkgo biloba (leaves), Fucus (thallus), ginseng (root), Eleutherococcus (root), caraway (seed), goldenrod (herb), everlasting flower (flower), blackcurrant (leaves and berries), glucomannan (tuber) and opunzia (cladophylls).

10. Method as claimed in claim 1, wherein the substances able to exercise effects on the body mediated by mechanical or physical effects are gums and mucilages.

11. Method as claimed in claim 10, wherein the gums and mucilages are extracted from plants chosen from French/blond psyllium (Plantago arenaria/Plantago ovata, seed), ribwort plantain (Plantago lanceolata), mallow (leaves, flowers), marshmallow (root), linden (flowers and leaves), flax (seed), barley (seed), senna (leaves and fruit), borage (leaves), cinnamon (bark), elder (flowers), tragacanth (gum), Sterculia (gum), acacia (gum), guar (gum) and xanthan (gum).

12. Method as claimed in claim 1, wherein the substances able to exercise effects on the body mediated by mechanical or physical effects are terpene and phenol resins oleoresins and gum resins.)

13. Method as claimed in claim 12, wherein the resins are extracted from propolis, benzoin, turpentine, balsam of Peru, balsam of Tolu, paprika, lentisk or mastic tree, myrrh, incense, ginger (root), caraway (seeds), Grindelia (flower head), asafoetida and lignum vitae.

14. Method as claimed in claim 1, wherein the substances able to exercise effects on the body mediated by mechanical or physical effects are lipids, triglycerides, waxes or fatty acids.

15. Method as claimed in claim 14, wherein the lipids, triglycerides, fatty acids or waxes are extracted from cocoa (seed), flax (seed), borage (seed), evening primrose (seed), argan (berries), almond (seed), wheat (seed), hemp (seed), blackcurrant (seed), anise (seed), caraway (fruit), cardamom (seed), coriander (seed), fennel (seed), blessed milk thistle (seed), everlasting flower (flower head) and arnica (flower).

16. Method as claimed in claim 1, wherein the biological test conducted at stage b) is a housekeeping gene expression test.

17. Method as claimed in claim 1, wherein the biological test conducted at stage b) is a test for inhibition of release of inflammation mediators from fibroblast cultures.

18. Medical devices obtainable by the methods claimed in claim 1.
